(19) United States
(12) Patent Application Publication
(10) Pub. No.: US 2010/0113527 A1
(43) Pub. Date: May 6, 2010

(54) CRYSTALLINE FORMS OF DEXLANSOPRAZOLE

(75) Inventors: Jacob RENDELL, Lehavim (IL); Ariel MITTELMAN, Elad (IL); Rinat MOSHKOVITS-KAPTSAN, Raanana (IL); Sergei FINE, Lahavim (IL); Ana KWOKAL, Zagreb (HR); Tamas KOLTAI, Netanya (IL)

Correspondence Address: KENYON & KENYON LLP, ONE BROADWAY, NEW YORK, NY, 10004, US

(73) Assignee: Teva Pharmaceutical Industries Ltd.

(21) Appl. No.: 12/571,100

(22) Filed: Sep. 30, 2009

Related U.S. Application Data
(60) Provisional application No. 61/101,318, filed on Sep. 30, 2008.
(60) Provisional application No. 61/105,904, filed on Oct. 16, 2008.
(60) Provisional application No. 61/118,212, filed on Nov. 26, 2008.
(60) Provisional application No. 61/146,465, filed on Jan. 22, 2009.
(60) Provisional application No. 61/224,340, filed on Jul. 9, 2009.
(60) Provisional application No. 61/106,032, filed on Oct. 16, 2008.

(51) Int. Cl. A61K 31/4439; C07D 401/12; A61P 1/00

(52) U.S. Cl. 514/338; 546/273.7

(57) ABSTRACT

Provided are crystalline forms of dexLansoprazole, as well as processes for the preparation thereof.

### CROSS-REFERENCE TO RELATED APPLICATIONS

The present invention claims the benefit of the following U.S. Provisional Patent Application Nos.: 61/101,318, filed Sep. 30, 2008; 61/105,904, filed Oct. 16, 2008; 61/118,212, filed Nov. 26, 2008; 61/146,465, filed Jan. 22, 2009; 61/224,340, filed Jul. 9, 2009; and 61/106,032, filed Oct. 16, 2008. The contents of these applications are incorporated.

### FIELD OF THE INVENTION

The invention encompasses crystalline forms of dexLansoprazole, as well as processes for the preparation thereof.

### BACKGROUND OF THE INVENTION

Lansoprazole, ((2-[[[3-methyl-4-(2,2,2-trifluoro-ethoxy)-2-pyridinyl]methyl]sulfinyl]-1H-benzimidazole) a substituted Benzimidazole, is an inhibitor of gastric (H⁺+K⁺)-ATPase. Lansoprazole per se is protected by U.S. Pat. No. 4,628,098 owned by Takeda, and has the following chemical formula:

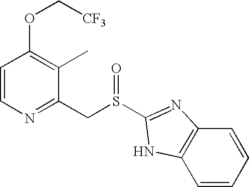

Lansoprazole is a racemic mixture of Lansoprazole enantiomers and was marketed by TAP Pharmaceutical Products under the trade name Prevacid® (in the US and Canada). dexLansoprazole, {(R)-2-[(3-methyl-4-(2,2,2-trifluoroethoxy)pyridin-2-yl)methylsulfinyl]-1H-benzoimidazole}, is the (R)-enantiomer of Lansoprazole. Takeda Pharmaceuticals has recently launched (US, May 2009) dexLansoprazole (KAPIDEX®). dexLansoprazole is a proton-pump inhibitor that suppresses gastric acid secretion by specific inhibition of the (H⁺, K⁺) ATPase in the gastric parietal cell. By acting specifically on the proton pump, dexLansoprazole blocks the final step of acid production, and used for the treatment of erosive esophagitis, heartburn and non-erosive gastro-esophageal reflux disease (GERD). The commercially available dexLansoprazole (KAPIDEX®) is based on a dual delayed release technology that delivers the drug in two separate releases, and has been approved by the U.S. FDA on Jan. 30, 2009.

dexLansoprazole has the following structure:

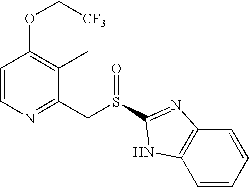

U.S. Pat. No. 5,948,789 describes a method for preparing dexLansoprazole.

PCT publications no. WO 2004/083200 (“WO '200”), WO 2000/78745 (“WO '745”), WO 2001/87874 (“WO '874”), WO 2002/044167 (“WO '167”) and US publication no. 2006/057195 (“US '195”) refer to crystalline forms of dexLansoprazole as well as to its amorphous form. PCT publication no. WO 09/088,857 (“WO '857”) describes crystalline hydrates and solvates of dexLansoprazole.

PCT publication no. WO 92/08716 (“WO '716”) described two Lansoprazole diastereomers, which undergo separation by chromatography and multiple re-crystallizations. The R-enantiomer is later recovered by acid hydrolysis.

PCT publication no. WO 96/02535 (“WO '535”) describes a process for preparing dexLansoprazole in a liquid phase. The process results in a low yield.

The invention relates to the solid state physical properties of dexLansoprazole. These properties can be influenced by controlling the conditions under which dexLansoprazole is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally administered active ingredient can reach the patient's bloodstream. The rate of dissolution is also a consideration in formulation syrups, elixirs, and other liquid medicaments. The solid state form of a compound can also affect its behavior on compaction and its storage stability.

These practical physical characteristics are influenced by the conformation and orientation of molecules in the unit cell, which define a particular polymorphic form of a substance. The polymorphic form can give rise to thermal behavior different from that of the amorphous material or another polymorphic form. Thermal behavior is measured in the laboratory by such techniques as capillary melting point, thermogravimetric analysis (“TGA”), and differential scanning calorimetry (“DSC”) and can be used to distinguish some polymorphic forms from others. A particular polymorphic form can also give rise to distinct spectroscopic properties that can be detectable by powder X-ray crystallography, solid state ¹³C NMR spectroscopy, and infrared spectrometry.

Generally, a crystalline solid has improved chemical and physical stability over the amorphous form, and forms with low crystallinity. Crystalline forms may also exhibit improved solubility, hygroscopicity, bulk properties, and/or flowability.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

There is a need in the art for additional forms of dexLansoprazole and processes for their preparation.

### SUMMARY OF THE INVENTION

In one embodiment, the present invention encompasses a crystalline form of dexLansoprazole, denominated Form X, characterized by data selected from the group consisting of: a PXRD pattern having peaks at about: 6.8, 12.2, 16.4, 17.8, 20.4 and 22.4±0.2 degrees 2-theta; and a PXRD pattern substantially as depicted in FIGS. 1 and 2.

In another embodiment, the invention encompasses a crystalline form X of dexLansoprazole further characterized by data selected from the group consisting of: a PXRD pattern having peaks at about: 6.8, 12.2, 14.4, 16.4, 17.8 and 20.4±0.2 degrees 2-theta; a DSC thermogram having an endothermic peak in the range of about 40° C. to about 90° C.; a weight loss (when heating to a temperature of about 100° C.) of less than about 2% as measured by TGA; a TGA pattern as depicted in FIG. 3; a DSC pattern as depicted in FIG. 4; and combinations thereof.

In yet another embodiment, the invention encompasses a process for preparing the crystalline Form X of dexLansoprazole comprising: dissolving dexLansoprazole in ethanol (“EtOH”), adding aqueous ammonium hydroxide to the solution to obtain a precipitate; removing the resulting precipitate; evaporating the filtrate to obtain a solid, and drying the solid.

In one embodiment, the invention encompasses a crystalline form of dexLansoprazole, denominated Form XI, characterized by data selected from the group consisting of: an X-ray powder diffraction pattern having peaks at about: 11.6 and 32.3±0.2 degrees 2-theta and at least three more peaks selected from the group consisting of peaks at about: 6.7, 12.1, 14.5, 18.7, 20.0, 22.0 and 23.6±0.2 degrees 2-theta; an X-ray powder diffraction pattern having peaks at about: 6.7, 14.5, 18.7 and 20.0±0.2 degrees 2-theta; an X-ray diffraction pattern substantially as depicted in any one of FIGS. 5-7; and combinations thereof.

In another embodiment, the invention encompasses the crystalline form XI of dexLansoprazole further characterized by data selected from the group consisting of: a DSC thermogram having an endothermic peak in the range of about 80° C. to about 150° C.; a TGA pattern as depicted in FIG. 8; a DSC pattern as depicted in FIG. 8; an X-ray powder diffraction pattern having peaks at about 11.6, 18.7, 22.0, 23.6 and 32.3±0.2 degrees 2-theta; an X-ray powder diffraction pattern having peaks at about 11.6, 12.1, 14.5, 20.0 and 32.3±0.2 degrees 2-theta; an X-ray powder diffraction pattern having peaks at about 6.7, 14.5, 17.8, 18.7, 20.0, 22.0, 29.6, 32.3 and 36.8±0.2 degrees 2-theta; and combinations thereof.

In yet another embodiment, the invention encompasses a process for preparing the crystalline Form XI of dexLansoprazole by crystallizing it from a mixture of ethyleneglycol and water.

In one embodiment, the present invention encompasses a crystalline form of dexLansoprazole denominated Form XII characterized by data selected from the group consisting of: an X-ray powder diffraction pattern having peaks at about 5.5, 13.2 and 19.7±0.2 degrees 2-theta and at least two more peaks selected from the group consisting of peaks at about: 7.0, 16.6, 17.9, 20.3, 21.2, 22.5 and 26.1±0.2 degrees 2-theta; an X-ray powder diffraction pattern having peaks at about 5.5, 13.2, 19.7 and 21.2±0.2 degrees 2-theta; an X-ray diffraction pattern substantially as depicted in FIG. 9; and combinations thereof.

In another embodiment, the invention encompasses the crystalline form XII of dexLansoprazole further characterized by data selected from the group consisting of: an X-ray powder diffraction pattern having peaks at about 5.5, 7.0, 13.2, 16.6 and 19.7±0.2 degrees 2-theta; an X-ray powder diffraction pattern having peaks at about 5.5, 13.2, 19.7, 21.2 and 22.5±0.2 degrees 2-theta; an X-ray powder diffraction pattern having peaks at about 5.5, 7.0, 13.2, 16.6, 19.7, 20.3, 21.2, 22.5 and 26.1±0.2 degrees 2-theta; and combinations thereof.

In yet another embodiment, the invention further encompasses a process for preparing the crystalline Form XII of dexLansoprazole by crystallizing it from a mixture of propyleneglycol and water.

In one embodiment, the present invention encompasses a crystalline form of dexLansoprazole, denominated Form XIII, characterized by data selected from the group consisting of: an X-ray powder diffraction pattern having peaks at about 14.4 and 19.2±0.2 degrees 2-theta and at least two more peaks selected from the group consisting of peaks at about: 18.4, 18.8, 20.6, 23.8 and 26.6±0.2 degrees 2-theta; an X-ray powder diffraction pattern having peaks at about: 6.7, 12.2, 13.4, 14.4 and 17.9±0.2 degrees 2-theta; an X-ray powder diffraction pattern having peaks at about: 6.7, 12.2, 14.4, 18.8 and 20.7±0.2 degrees 2-theta; an X-ray diffraction pattern substantially as depicted in any one of FIGS. 10-13; and combinations thereof.

In another embodiment, the present invention encompasses the crystalline form XIII of dexLansoprazole further characterized by data selected from the group consisting of: an X-ray powder diffraction pattern having peaks at about 14.4, 18.4, 19.2, 20.6 and 23.8±0.2 degrees 2-theta; an X-ray powder diffraction pattern having peaks at about 14.4, 18.4, 18.8, 19.2 and 26.6±0.2 degrees 2-theta; an X-ray powder diffraction pattern having peaks at about 6.7, 12.2, 13.4, 14.4, 17.9, 18.4, 18.8, 19.2, 19.8, 20.3 and 20.7±0.2 degrees 2-theta; and combinations thereof.

In yet another embodiment, the present invention further encompasses a process for preparing the crystalline Form XIII of dexLansoprazole by combining it with EtOH.

In one embodiment, the present invention encompasses a crystalline form of dexLansoprazole denominated Form XIV characterized by data selected from the group consisting of: an X-ray powder diffraction pattern having peaks at about 8.8 and 10.2±0.2 degrees 2-theta and at least two more peaks selected from the group consisting of peaks at about: 13.2, 13.5, 15.5, 15.9, 18.4 and 22.3±0.2 degrees 2-theta; an X-ray powder diffraction pattern having peaks at about 15.5, 15.9, 20.4, 22.3 and 22.8; an X-ray diffraction pattern substantially as depicted in FIG. 14; and combinations thereof.

In another embodiment, the present invention encompasses the crystalline form XIV of dexLansoprazole further characterized by data selected from the group consisting of: a DSC thermogram having endothermic peaks in a range of about 40° C. to about 60° C. and in the range of about 110° C. to about 140° C.; a DSC pattern as depicted in FIG. 15; an X-ray powder diffraction pattern having peaks at about 8.8, 10.2, 13.5, 15.9 and 18.4±0.2 degrees 2-theta; an X-ray powder diffraction pattern having peaks at about 8.8, 10.2, 13.2, 15.5 and 22.3±0.2 degrees 2-theta; an X-ray powder diffraction pattern having peaks at about 8.8, 13.5, 15.5, 15.9, 18.4, 20.4, 22.3, 22.8 and 25.8±0.2±0.2 degrees 2-theta; and combinations thereof.

In yet another embodiment, the invention encompasses a process for preparing the crystalline Form XIV of dexLansoprazole comprising: dissolving dexLansoprazole in EtOH and ammonium hydroxide to obtain a solution; adding diisopropyl ether to the solution to obtain a precipitate; removing the resulting precipitate; evaporating the filtrate to obtain a solid, and drying the solid.

In another embodiment, the invention encompasses a pharmaceutical formulation comprising a therapeutically effective amount of at least one of the above-described forms of dexLansoprazole, and at least one pharmaceutically acceptable excipient.

In yet another embodiment, the invention encompasses a process for preparing a pharmaceutical formulation comprising combining at least one of the above-described forms of dexLansoprazole, with at least one pharmaceutically acceptable excipient.

In one embodiment, the invention encompasses the use of a pharmaceutical formulation comprising a therapeutically effective amount of at least one of the above-described crystalline forms of dexLansoprazole and at least one pharmaceutically acceptable excipient in the manufacture of a pharmaceutical composition.

In another embodiment, the invention encompasses methods of treating or preventing erosive oesophagitis and non-erosive gastroesophageal reflux comprising administering a pharmaceutical formulation comprising a therapeutically effective amount of at least one of the above-described forms of dexLansoprazole, and at least one pharmaceutically acceptable excipient to a patient in need thereof.

### BRIEF DESCRIPTION OF THE DRAWINGS

FIG. 1 illustrates an X-ray powder diffraction pattern of Form X of dexLansoprazole.

FIG. 2 illustrates an X-ray powder diffraction pattern of form X of dexLansoprazole compared with the same form X after a week at 60% relative humidity.

FIG. 3 illustrates a DSC analysis of form X of dexLansoprazole.

FIG. 4 illustrates a TGA analysis of form X of dexLansoprazole.

FIG. 5 illustrates an X-ray powder diffraction pattern of Form XI of dexLansoprazole obtained according to example 2.

FIG. 6 illustrates an X-ray powder diffraction pattern of Form XI of dexLansoprazole obtained according to example 4.

FIG. 7 illustrates an X-ray powder diffraction pattern of Form XI of dexLansoprazole obtained according to example 3.

FIG. 8 illustrates DSC and TGA patterns of Form XI of dexLansoprazole.

FIG. 9 illustrates an X-ray powder diffraction pattern of Form XII of dexLansoprazole.

FIG. 10 illustrates an X-ray powder diffraction pattern of Form XIII of dexLansoprazole obtained according to example 6.

FIG. 11 illustrates an X-ray powder diffraction pattern of Form XIII of dexLansoprazole obtained according to example 7.

FIG. 12 illustrates an X-ray powder diffraction pattern of Form XIII of dexLansoprazole obtained according to example 8.

FIG. 13 illustrates an X-ray powder diffraction pattern of Form XIII of dexLansoprazole obtained according to example 19.

FIG. 14 illustrates an X-ray powder diffraction pattern of Form XIV of dexLansoprazole.

FIG. 15 illustrates DSC pattern of Form XIV of dexLansoprazole.

### DETAILED DESCRIPTION OF THE INVENTION

The present invention provides novel crystalline forms of dexLansoprazole.

As used herein in connection with a measured quantity, the term “about” refers to that variation in the measured quantity as would be expected by the skilled artisan performing the measurement and exercising a level of care commensurate with the objective of the measurement and the precision of the measuring apparatus being used.

As used herein, the term “room temperature” refers to a temperature of about 15° C. to about 30° C., preferably about 20° C. to about 25° C.

As used herein, the term “reduced pressure” or “vacuum” refers to a pressure of about 760 mmHg or less, preferably, about 100 mmHg to about 2 mmHg.

As used herein, the term “over night” refers to a period of time of about 6 hours to about 24 hours, preferably, of about 10 to about 20 hours.

As used herein, the term “RH” or “rh” refers to relative humidity. The relative humidity in air may be described as the ratio of the partial pressure of water vapor in the mixture to the saturated vapor pressure of water at a prescribed temperature.

As used herein, the term “e.e.” refers to enantiomeric excess, and can be generally defined as the difference between the mole fraction of each enantiomer in a mixture where one enantiomer exists in excess over the other. More specifically in relation to this invention, enantiomeric excess can be defined as: (mole fraction of the (R)-enantiomer) minus (mole fraction of the (S)-enantiomer).

As used herein, the term “optically pure” refers to a compound having more than about 95% e.e, preferably, more than about 98% e.e, more preferably, more than about 99% e.e., more preferably, more than about 99.7% e.e, most preferably, more than about 99.9% e.e.

As used herein, the term “optically enriched” refers to a compound with an improved optical purity when compared with a previous sample of the compound.

As used herein, the term “solid phase” refers to one of the three fundamental structural phases of matter in which the cohesive force of matter is strong enough to keep the molecules or atoms in the given positions, restraining the thermal mobility.

As used herein in connection with form X, the term “stable” refers to stability against transformation, in which no more than about 20% of a polymorphic form transforms to another form under no less than about 60% relative humidity at about room temperature. When made according to the preferred embodiments of the present invention, less than 20% of form X transforms to any other form upon exposure to a RH of 60% at room temperature for a period of 7 days.

As used herein, the term “absolute” refers to a solvent containing about 1% (weight/weight percentage) or less of water, preferably, 0.5% or less of water, more preferably, 0.25% or less of water, most preferably, 0.15% or less of water.

As used herein, the term “PXRD” refers to Powder X-ray Diffraction.

As used herein, the term “DLNP” refers to dexLansoprazole.

As used herein, the term “DSC” refers to Differential Scanning Calorimetric.

As used herein, the term “TGA” refers to Thermo Gravimetric Analysis.

As used herein the term “crystalline purity,” refers to a particular crystalline form of a compound in a sample which may contain amorphous form of the compound, one or more other crystalline forms of the compound other than the crystalline form of the compound of this invention, or a mixture thereof wherein the particular form of the compound is present in an amount of at least about 80%, preferably at least about 95%, most preferably at least about 99% crystalline.

As used herein, the term volume (“V”) refers to ml per gram. For example, 30 V of solvent means 30 ml solvent per one gram of solid/compound.

The references WO '200 and US '195 describe several crystalline forms of dexLansoprazole. The following table summarizes the characteristic X-ray powder diffraction pattern peaks, in D-spacing values, of some of the forms, described therein (all values are in Angstrom):

| | Form III | Form III | | | |
|---|---|---|---|---|---|
| Form II | (sesquihydrate) | (sesquihydrate) | form IV | Form V | Form VI |
| (anhydrous) | (According to WO '200) | (According to US '195) | (hydrate) | (hydrate) | (hemihydrate) |
| 11.68 | 13.22 | 8.91 | 5.85 | 8.33 | 9.50 |
| 6.77 | 9.61 | 8.07 | 4.70 | 6.63 | 8.73 |
| 5.84 | 8.87 | 6.62 | 4.35 | 5.86 | 8.31 |
| 5.73 | 8.05 | 6.00 | 3.66 | 4.82 | 5.57 |
| 4.43 | 6.61 | 5.92 | 3.48 | | 5.18 |
| 4.09 | 5.92 | 5.66 | | | 4.80 |
| 3.94 | 5.65 | 5.04 | | | 4.20 |
| 3.89 | 5.02 | 4.51 | | | |
| 3.69 | 4.49 | | | | |
| 3.41 | 3.50 | | | | |
| 3.11 | 3.00 | | | | |

The invention addresses a need in the art by providing additional crystalline forms of dexLansoprazole, as well as processes for their preparation.

Unless otherwise indicated, the dexLansoprazole starting material used in the processes of the present invention may be obtained by any method known in the art, for example, as described in U.S. Pat. No. 5,948,789, wherein 2-[[[3-methyl-4-(2,2,2-trifluoroethoxy)-2-pyridinyl]-methyl]thio]-1H-benzimidazole is dissolved in toluene; water, (+)-diethyl L-tartrate and titanium(IV) isopropoxide is added to the solution to obtain a mixture; the mixture is stirred for 60 minutes at 50° C. and then cooled to room temperature; N,N-diisopropylethylamine and cumene hydroperoxide (80%) are added and stirred for 16 h at room temperature; toluene is added to the mixture and the dexLansoprazole is recovered using extraction, evaporation and flash chromatography. Unless otherwise specifically indicated, the starting material for the processes preparing crystalline dexLansoprazole described herein is form II of dexLansoprazole.

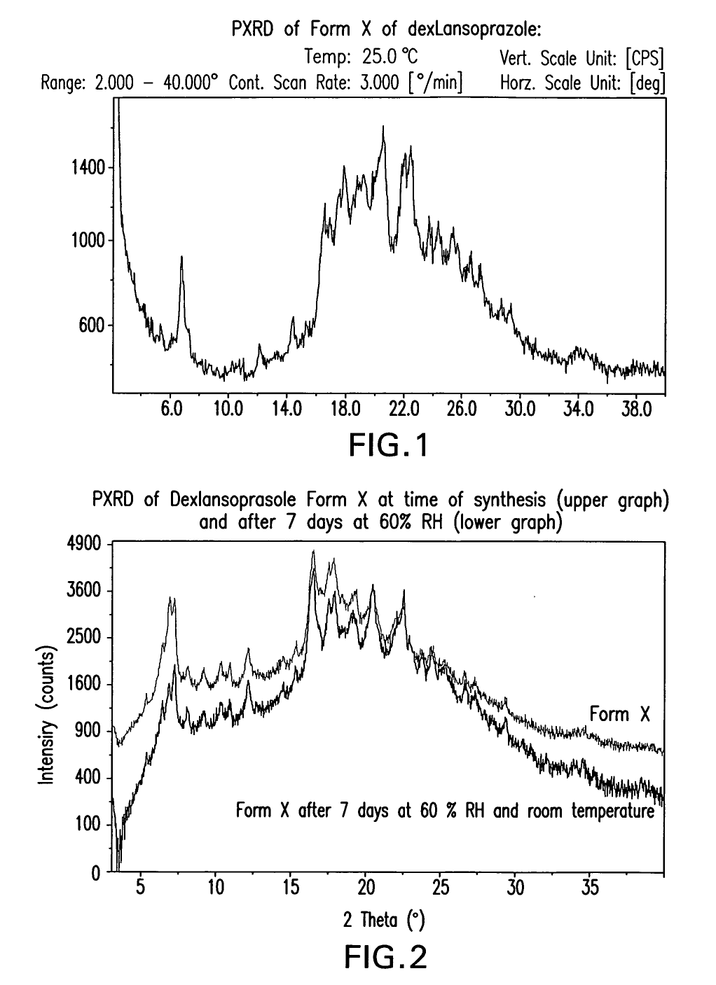

The invention encompasses a crystalline form of dexLansoprazole, denominated Form X, characterized by data selected from the group consisting of: a PXRD pattern having peaks at about: 6.8, 12.2, 16.4, 17.8, 20.4 and 22.4±0.2 degrees 2-theta; a PXRD pattern substantially as depicted in FIGS. 1 and 2; and combinations thereof.

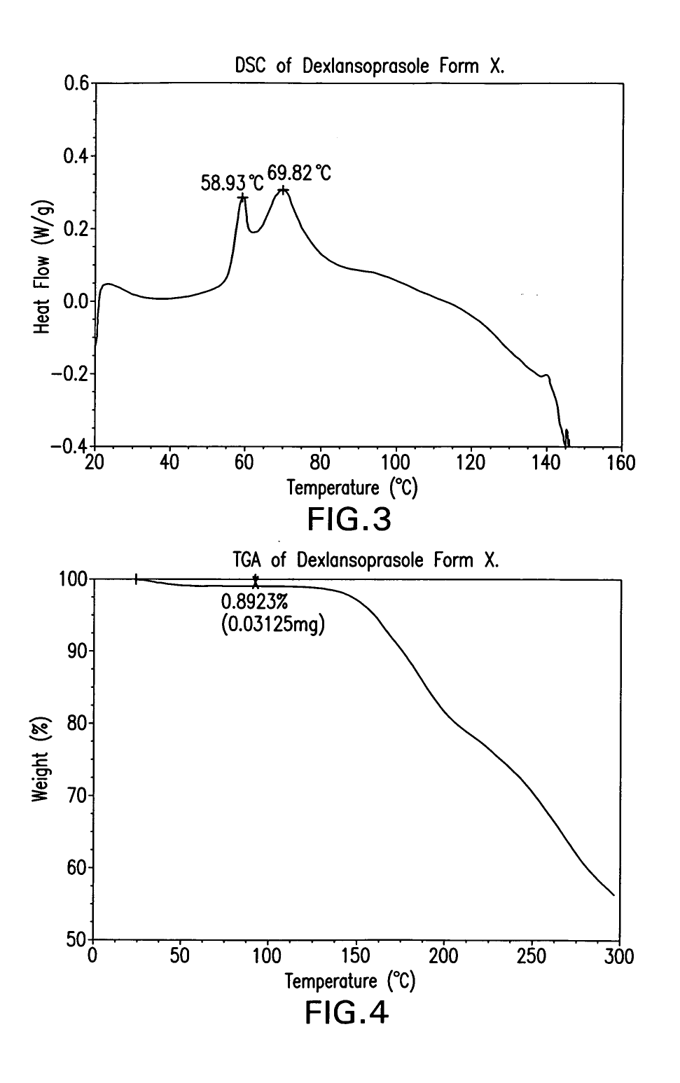

The crystalline form X of dexLansoprazole may be further characterized by data selected from the group consisting of: a PXRD pattern having peaks at about: 6.8, 12.2, 14.4, 16.4, 17.8 and 20.4±0.2 degrees 2-theta; a DSC thermogram having an endothermic peak at about 40° C. to about 90° C.; a weight loss (when heating to a temperature of about 100° C.) of less than about 2% as measured by TGA; a TGA pattern as depicted in FIG. 3; a DSC pattern as depicted in FIG. 4; and combinations thereof.

Preferably the crystalline form X of dexLansoprazole has a crystalline purity of at least 80%, more preferably at least 90%, more preferably at least 95%, most preferably at least 99% by weight.

As shown in FIG. 2, form X retains its PXRD pattern after 7 days at a relative humidity of 60%, demonstrating the stability of form X at a relative humidity of 60% at room temperature for a period of at least 7 days.

The invention encompasses a process for preparing the crystalline Form X of dexLansoprazole comprising: dissolving dexLansoprazole in ethanol (“EtOH”), adding aqueous ammonium hydroxide to the solution to obtain a precipitate; removing the resulting precipitate; evaporating the filtrate to obtain a solid, and drying the solid.

Preferably, the addition of ammonium hydroxide is done at about room temperature. Preferably, the concentration of the ammonium hydroxide is between about 15% to about 30%, more preferably, about 20% to about 27%, most preferably, about 25%.

Typically, after the ammonium hydroxide addition, a solution is obtained. The obtained solution is preferably maintained while stirring. Preferably, stirring is done for a period of about 15 minutes to about 2 hours, more preferably, for about 30 minutes. Typically a precipitate is formed in the solution, which is removed prior to evaporating the solvent from the solution. Preferably, removing the precipitate is done by filtration. Preferably, the obtained filtrate is dried by evaporation to obtain a solid form.

Preferably, drying is at a temperature of about 20° C. to about 30° C., more preferably at about 25° C. Preferably, drying is performed for about 12 hours to about 24 hours, more preferably, for about 16 hours.

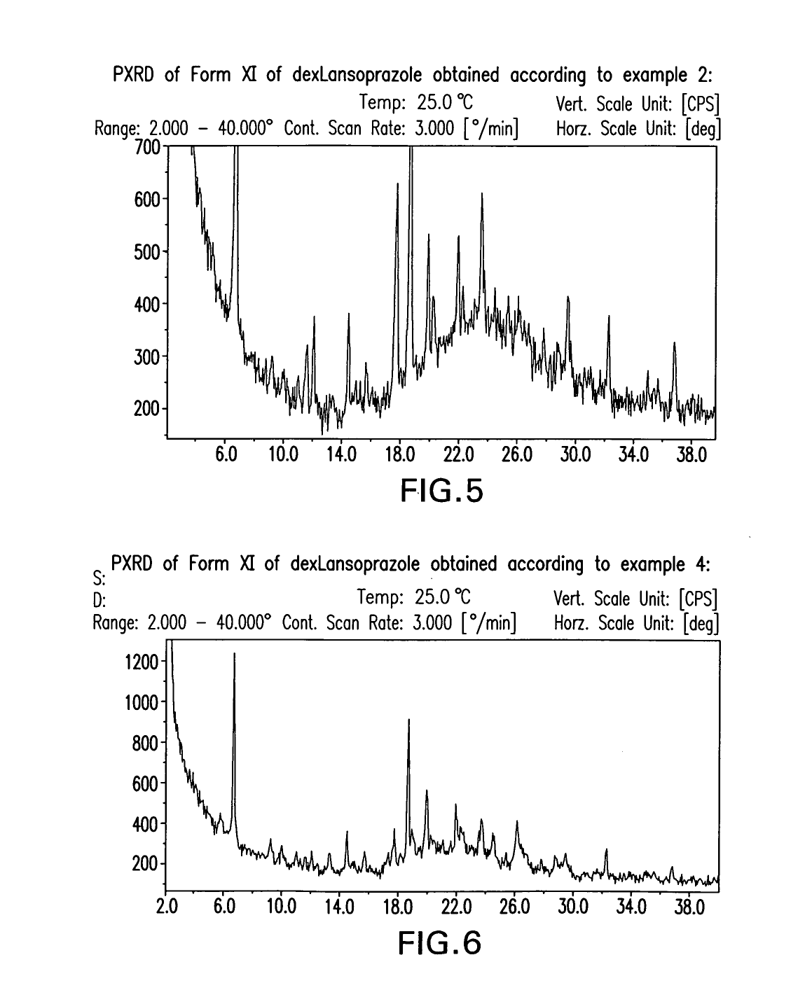

The invention also encompasses a crystalline form of dexLansoprazole, denominated Form XI, characterized by data selected from the group consisting of: an X-ray powder diffraction pattern having peaks at about: 11.6 and 32.3±0.2 degrees 2-theta and at least three more peaks selected from the group consisting of peaks at about: 6.7, 12.1, 14.5, 18.7, 20.0, 22.0 and 23.6±0.2 degrees 2-theta; an X-ray powder diffraction pattern having peaks at about: 6.7, 14.5, 18.7 and 20.0±0.2 degrees 2-theta; an X-ray diffraction pattern substantially as depicted in any one of FIGS. 5-7; and combinations thereof.

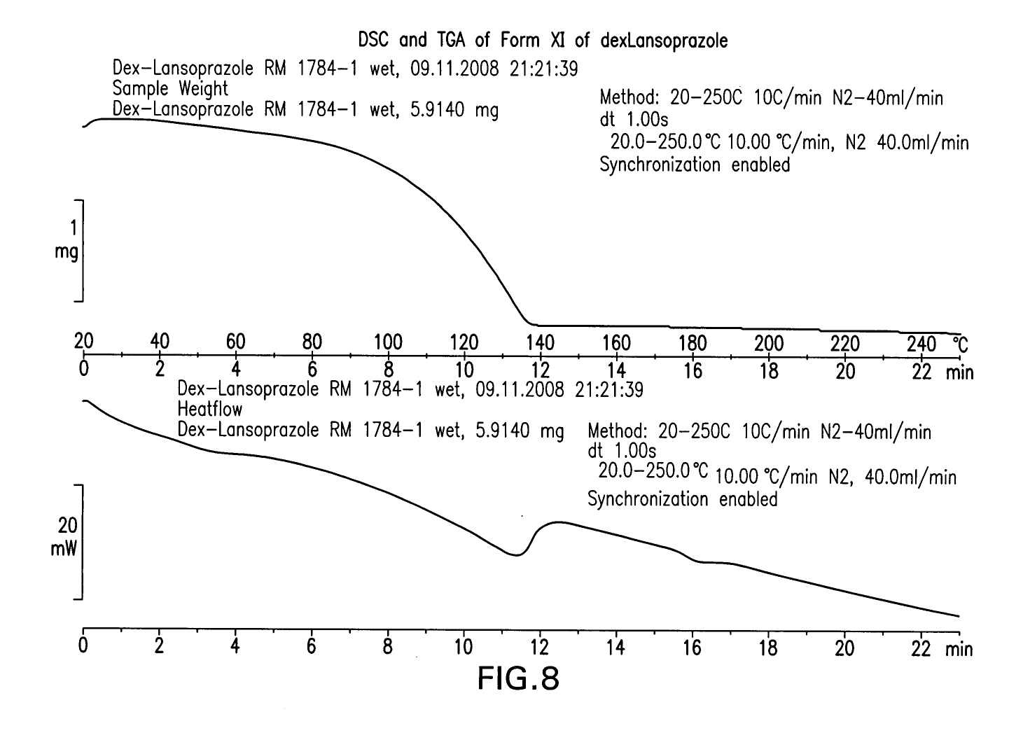

Optionally, the crystalline form XI of dexLansoprazole may be further characterized by data selected from the group consisting of: a DSC thermogram having an endothermic peak in the range of about 80° C. to about 150° C.; a TGA pattern as depicted in FIG. 8; a DSC pattern as depicted in FIG. 8; an X-ray powder diffraction pattern having peaks at about 11.6, 18.7, 22.0, 23.6 and 32.3±0.2 degrees 2-theta; an X-ray powder diffraction pattern having peaks at about 11.6, 12.1, 14.5, 20.0 and 32.3±0.2 degrees 2-theta; an X-ray powder diffraction pattern having peaks at about 6.7, 14.5, 17.8, 18.7, 20.0, 22.0, 29.6, 32.3 and 36.8±0.2 degrees 2-theta; and combinations thereof.

Preferably the crystalline form XI of dexLansoprazole has a crystalline purity of at least 80%, more preferably at least 90%, more preferably at least 95%, most preferably at least 99% by weight.

The present invention encompasses a process for preparing the crystalline Form XI of dexLansoprazole by crystallizing it from a mixture of ethyleneglycol and water. Preferably, the crystallization comprises: dissolving dexLansoprazole in ethyleneglycol; and adding water to the solution to obtain a precipitate.

Optionally, the ethyleneglycol is added to dexLansoprazole while stirring. Preferably, the amount of ethylenglycol added is about 3 ml to about 15 ml per gram of dexLansoprazole, more preferably, about 5 ml to about 10 ml per gram of dexLansoprazole.

Preferably, the ratio of water to ethylenglycol is about 1:1 to about 10:1, more preferably, about 2:1.

Prior to the water addition, a stirring step may be performed. Preferably, stirring is at about room temperature. Preferably, stirring is for a period of about 6 hours to about 24 hours, preferably of about 12 hours to about 18 hours. Preferably, stirring is done at about room temperature.

Optionally, following the water addition, a heating step is performed followed by a cooling step to obtain a slurry. Preferably, heating is to a temperature of about 70° C. to about 90° C., preferably about 80° C. Preferably, prior to the cooling step, a stirring step is performed for about 1 hour. Preferably, cooling is to about room temperature. The resulting slurry may be further stirred, and filtered to obtain the crystalline form XI.

Alternatively, the crystallization comprises: dissolving dexLansoprazole in ethylenglycol; adding hexane; and subsequently adding water to obtain a precipitate.

Preferably, the amount of ethylene glycol is of about 1 to about 30 ml per gram of dexLansoprazole, more preferably, about 1 to about 10 ml per gram of dexLansoprazole, more preferably, about 1 to about 5 ml per gram of dexLansoprazole, most preferably, about 2.5 to about 4 ml per gram of dexLansoprazole.

Preferably, the hexane is added at about room temperature.

Preferably, the amount of hexane is of about 1 to about 10, more preferably, about 1 to about 5, most preferably, about 2 to about 4 ml per gram of dexLansoprazole.

After the addition of hexane, a solution is obtained. Optionally, the solution obtained after the addition of hexane is cooled and heated thereafter. Cooling is preferably done to about −10° C. to about −30° C., preferably to about −20° C. Heating is preferably done to about room temperature.

Preferably, water is added at room temperature.

Preferably, the ratio of water to ethylenglycol is about 1:1 to about 10:1, more preferably, about 2:1.

After the water addition, a slurry is obtained. The slurry may be stirred, optionally at about room temperature for a period of about 6 hours to about 48 hours, more preferably, for a period of about 12 hours to about 18 hours, most preferably, for about 12 hours.

Preferably, the precipitate obtained in the process for preparing crystalline form XI above is isolated. The isolation may be done by filtration.

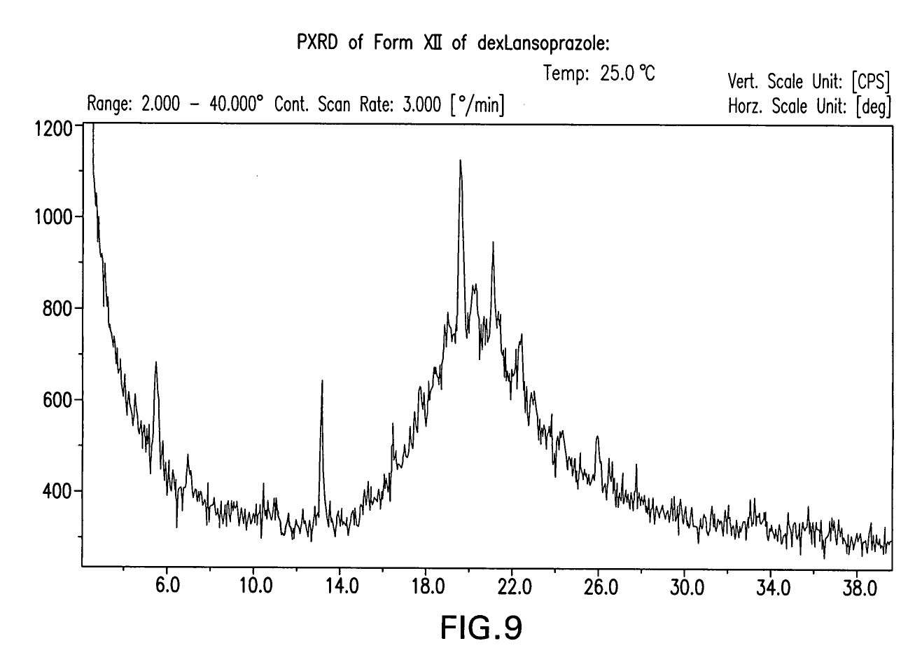

The present invention encompasses a crystalline form of dexLansoprazole denominated Form XII characterized by data selected from the group consisting of: an X-ray powder diffraction pattern having peaks at about 5.5, 13.2 and 19.7±0.2 degrees 2-theta and at least two more peaks selected from the group consisting of peaks at about: 7.0, 16.6, 17.9, 20.3, 21.2, 22.5 and 26.1±0.2 degrees 2-theta; an X-ray powder diffraction pattern having peaks at about 5.5, 13.2, 19.7 and 21.2±0.2 degrees 2-theta; an X-ray diffraction pattern substantially as depicted in FIG. 9; and combinations thereof.

Optionally, the crystalline form XII of dexLansoprazole may be further characterized by data selected from the group consisting of: an X-ray powder diffraction pattern having peaks at about 5.5, 7.0, 13.2, 16.6 and 19.7±0.2 degrees 2-theta; an X-ray powder diffraction pattern having peaks at about 5.5, 13.2, 19.7, 21.2 and 22.5±0.2 degrees 2-theta; an X-ray powder diffraction pattern having peaks at about 5.5, 7.0, 13.2, 16.6, 19.7, 20.3, 21.2, 22.5 and 26.1±0.2 degrees 2-theta; and combinations thereof.

Preferably the crystalline form XII of dexLansoprazole has a crystalline purity of at least 80%, more preferably at least 90%, more preferably at least 95%, most preferably at least 99% by weight.

The present invention further encompasses a process for preparing the crystalline Form XII of dexLansoprazole by crystallizing it from a mixture of propyleneglycol and water. Preferably, the crystallization comprises: dissolving dexLansoprazole in propyleneglycol; and adding water to the solution to obtain a precipitate.

Preferably, the dissolution step is done at about room temperature.

Preferably, the amount of propylene glycol is of about 1 to about 20 ml per gram of dexLansoprazole, more preferably, about 1 to about 10, more preferably, about 3 to about 5, most preferably, about 4 ml per gram of dexLansoprazole.

Preferably, the solution is maintained while stirring. Preferably, stirring is done at about room temperature, more preferably, at a temperature of about 18° C. to about 27° C. Stirring may be done for about 6 hours to about 48 hours, more preferably, for about 12 hours.

Preferably, the water addition is done at about room temperature.

Preferably, the ratio of water to propyleneglycol is about 1:1 to about 10:1, more preferably, about 2:1.

Typically, after the addition of water, a suspension is obtained. Preferably, the suspension is maintained while stirring. Preferably, stirring is for about 24 hours to about 48 hours, more preferably, for about 24 hours.

Preferably, the precipitate is isolated. The isolation may be done by filtration.

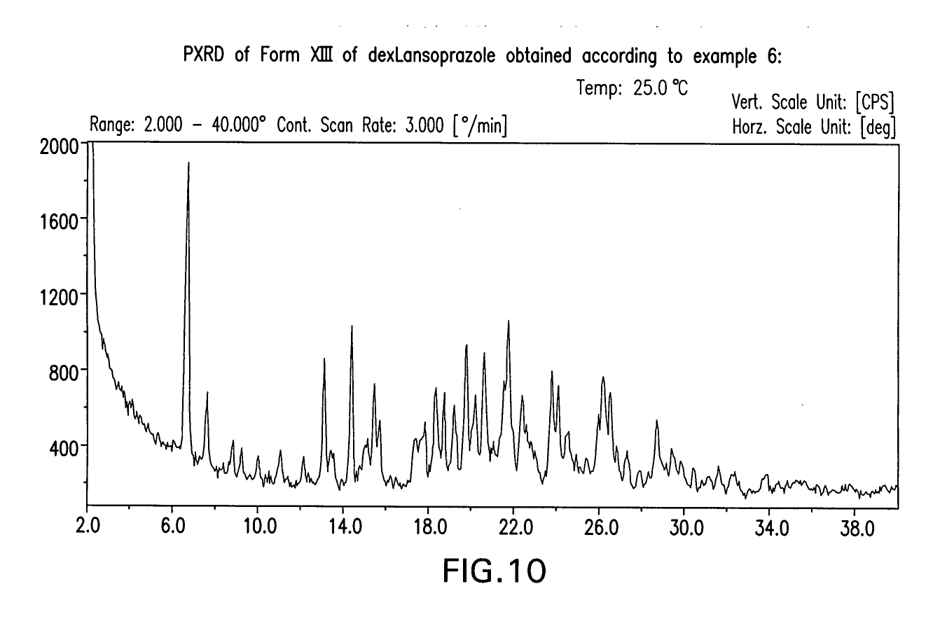

The invention also encompasses a crystalline form of dexLansoprazole, denominated Form XIII, characterized by data selected from the group consisting of: an X-ray powder diffraction pattern having peaks at about 14.4 and 19.2±0.2 degrees 2-theta and at least two more peaks selected from the group consisting of peaks at about: 18.4, 18.8, 20.6, 23.8 and 26.6±0.2 degrees 2-theta; an X-ray powder diffraction pattern having peaks at about: 6.7, 12.2, 13.4, 14.4 and 17.9±0.2 degrees 2-theta; an X-ray powder diffraction pattern having peaks at about: 6.7, 12.2, 14.4, 18.8 and 20.7±0.2 degrees 2-theta; an X-ray diffraction pattern substantially as depicted in any one of FIGS. 10-13; and combinations thereof.

Optionally, the crystalline form XIII of dexLansoprazole may be further characterized by data selected from the group consisting of: an X-ray powder diffraction pattern having peaks at about 14.4, 18.4, 19.2, 20.6 and 23.8±0.2 degrees 2-theta; an X-ray powder diffraction pattern having peaks at about 14.4, 18.4, 18.8, 19.2 and 26.6±0.2 degrees 2-theta; an X-ray powder diffraction pattern having peaks at about 6.7, 12.2, 13.4, 14.4, 17.9, 18.4, 18.8, 19.2, 19.8, 20.3 and 20.7±0.2 degrees 2-theta; and combinations thereof.

The crystalline form XIII of dexLansoprazole may be an ethanolate.

Preferably the crystalline form XIII of dexLansoprazole has a crystalline purity of at least 80%, more preferably at least 90%, more preferably at least 95%, most preferably at least 99% by weight.

A process for preparing the crystalline Form II of dexLansoprazole may comprise: combining dexLansoprazole with EtOH and ammonium hydroxide; heating; and adding diisopropyl ether to obtain a precipitate.

Optionally, the EtOH is absolute.

The present invention provides a process for preparing the crystalline form XIII of dexLansoprazole comprising: combining dexLansoprazole with EtOH and ammonium hydroxide; heating; adding diisopropyl ether; evaporating the filtrate to obtain a precipitate; and drying the precipitate.

Preferably, when the starting dexLansoprazole has a purity of about 75% to about 85%, or less, the addition of diisopropyl ether is optional.

Preferably, the amount of ethanol used is about 2 volumes to about 6 volumes.

Preferably, the ratio of ammonium hydroxide to ethanol is of about 1:6 to about 1:16.

Additional amounts of ethanol and ammonium hydroxide may be added.

Prior to the heating step, a precipitate may be formed, and thereafter removed to obtain a mother liquor. The mother liquor may be concentrated and treated again with additional amount of ethanol and ammonium hydroxide.

Preferably, heating is to a temperature of about RT to about 40° C., more preferably, to a temperature of about 30° C. to about 40° C., most preferably, to a temperature of about 35° C. to about 40° C. to obtain a solution.

Typically, following the heating step and prior to the addition of diisopropyl ether, the solution is cooled. Preferably, cooling is to about room temperature.

Preferably, the ratio of diisopropyl ether to ethanol is of about 5:1 to about 15:1.

After the addition of diisopropyl ether, further cooling is performed. Preferably, cooling is to a temperature of about 5° C. to about −20° C., more preferably, cooling is to a temperature of about 5° C. to about 0° C.

Typically, an initial precipitate is formed in the solution after the addition of diisopropyl ether and cooling of the solution, which is removed prior to evaporating the solvent from the solution. Preferably, removing this initial precipitate is done by filtration. Preferably, the obtained filtrate is dried by evaporation to obtain a precipitate.

The drying may be done in a vacuum oven at about room temperature to obtain form XIII. Preferably, drying is performed for about 12 hours to about 24 hours, more preferably, for about 16 hours.

The process described above provides a process for making a highly optically pure dexLansoprazole. Preferably, the optically pure dexLansoprazole obtained by the process described above has a purity of more than about 99% e.e. This process is performed in a solid phase and has several advantages over the previously known processes carried out in the liquid phase. The enrichment process in the solid phase is straight forward (filtration), since it avoids evaporations as done in the liquid phase and thus reduces the potential damage to the molecule in high temperatures as well as improving the yield. Additionally, the absence of solvent and the fact that the process does not have to be repeated several times, as in the liquid phase, makes the process cost efficient and environmentally friendly.

Optically purifying dexLansoprazole in the solid phase is superior to purification in the liquid phase. Optical purity can only reach a certain maximal level in the liquid phase whereas purification in the solid phase can attain higher levels of optical purity.

The precipitate obtained in the processes above for Form XIII may be further isolated. Typically, the isolation is by filtration.

The invention also encompasses a process for preparing Form XIII of dexLansoprazole comprising: drying Form II of dexLansoprazole. Drying may be done in a vacuum oven at about room temperature to obtain form XIII.

Preferably, drying is performed for about 12 hours to about 24 hours, more preferably, for about 16 hours.

Another process for preparing crystalline form XIII comprises: storing a dexLansoprazole selected from the group consisting: form X of dexLansoprazole, amorphous form of dexLansoprazole, form VI of dexLansoprazole, form III of dexLansoprazole and combinations thereof in the presence of ethanol.

Preferably, storing is for about 3 days to about 4 weeks, more preferably for about 3 weeks. Preferably, storing is at about room temperature.

Preferably, the ethanol is absolute.

Alternatively, the preparation of crystalline form XIII comprises: combining dexLansoprazole and ethanol to obtain a wet powder; and grinding the obtained mixture.

Preferably, the ethanol is absolute.

Preferably, grinding is for about 30 seconds to about 10 minutes, preferably about 30 seconds to about 5 minutes, most preferably, about 1 minute.

The preparation of crystalline form XIII may comprise: slurrying a dexLansoprazole selected from the group consisting of: form X of dexLansoprazole; amorphous dexLansoprazole; form III of dexLansoprazole; and mixtures thereof in ethanol.

Preferably, the ethanol is absolute.

The slurrying may be performed at a temperature of about 20° C. to about 30° C., more preferably, of about 23° C. to about 25° C.

Preferably, following the addition of ethanol, a stirring step is performed. Preferably, the stirring is for about 2 minutes to about 10 minutes, more preferably, for about 5 minutes.

Optionally, following the slurrying step, seeding with dexLansoprazole is performed to obtain form XIII of dexLansoprazole. Preferably, the seeding is done with amorphous dexLansoprazole.

Optionally, following seeding, a stirring step is performed. Preferably, the stirring is for about 5 minutes to about 30 minutes, more preferably, for about 10 minutes to about 15 minutes. Preferably, the stirring is at about room temperature.

Preferably, the obtained crystalline form is recovered. The recovery may be done by evaporation. Preferably, the evaporation is done at a temperature of about 20° C. to about 30° C., more preferably, of about 23° C. to about 25° C. Optionally, the obtained solid form is isolated. Preferably, the isolation is done by filtration.

Optionally, the slurring is done with ethanol saturated with ammonia gas or in a mixture with ammonium hydroxide.

This process may further comprise a stirring step. Preferably, the stirring is for about 5 minutes to about 30 minutes, more preferably, for about 10 minutes to about 15 minutes. Preferably, the stirring is at about room temperature.

This process further comprises a cooling step. Preferably, the cooling is to a temperature of about −5° C. to about −20° C., more preferably, to about −18° C. Preferably, the cooling is for about three days to about two weeks, more preferably, for about one week.

The obtained crystalline form is further isolated. Preferably, the isolation is done by filtration.

Another process for the preparation of crystalline form XIII may comprise: suspending a mixture of amorphous dexLansoprazole and form XIV of dexLansoprazole in ethanol. Preferably, the ethanol is added saturated with ammonia.

Preferably, the ethanol is absolute.

Preferably, the process further comprises a stirring step. Preferably, stirring is for about 5 minutes to about 30 minutes, more preferably, for about 10 minutes to about 15 minutes. Preferably, the stirring is at about 0° C. to about 10° C., more preferably, at about 0° C. to about 5° C.

Optionally, this process further comprises a cooling step. Preferably, the cooling is to a temperature of about −5° C. to about −20° C., more preferably, to about −18° C. Preferably, the cooling is for three days to about two weeks, more preferably, for about a week.

Preferably, the obtained solid form is isolated. Preferably, the isolation is done by centrifugation.

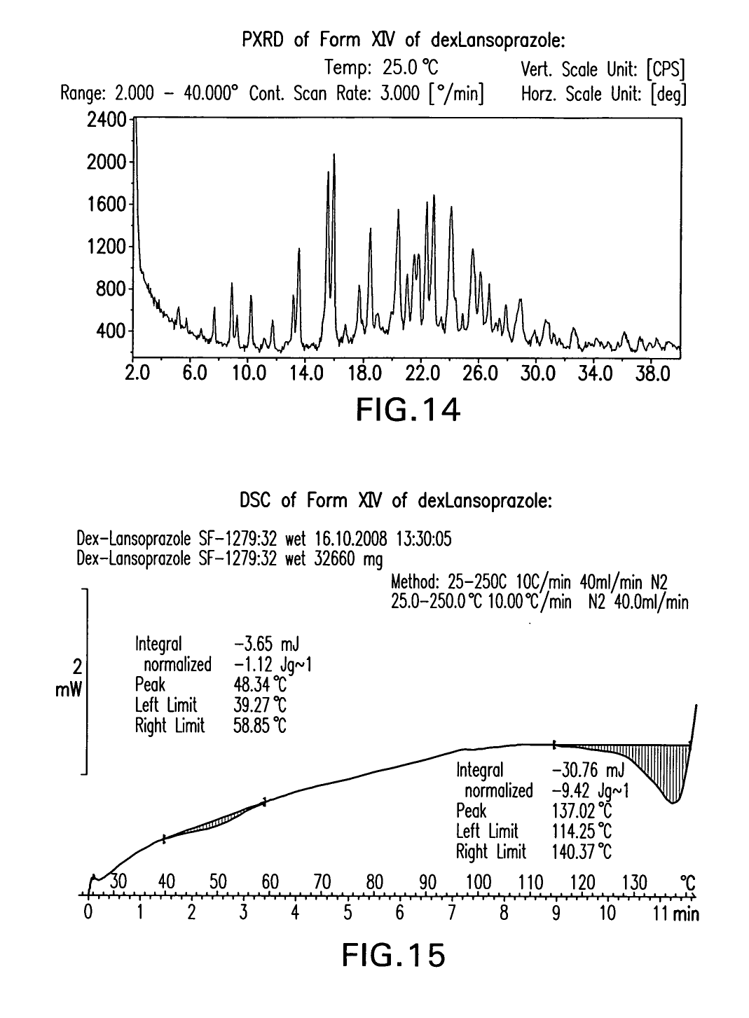

The invention encompasses a crystalline form of dexLansoprazole denominated Form XIV characterized by data selected from the group consisting of: an X-ray powder diffraction pattern having peaks at about 8.9 and 10.2±0.2 degrees 2-theta and at least two more peaks selected from the group consisting of peaks at about: 13.2, 13.5, 15.5, 15.9, 18.4 and 22.3±0.2 degrees 2-theta; an X-ray powder diffraction pattern having peaks at about 15.5, 15.9, 20.4, 22.3 and 22.8; an X-ray diffraction pattern substantially as depicted in FIG. 14; and combinations thereof.

Optionally, the crystalline form XIV of dexLansoprazole may be further characterized by data selected from the group consisting of: a DSC thermogram having endothermic peaks of about 40° C. to about 60° C. and of about 110° C. to about 140° C.; a DSC pattern as depicted in FIG. 15; an X-ray powder diffraction pattern having peaks at about 8.9, 10.2, 13.5, 15.9 and 18.4±0.2 degrees 2-theta; an X-ray powder diffraction pattern having peaks at about 8.9, 10.2, 13.2, 15.5 and 22.3±0.2 degrees 2-theta; an X-ray powder diffraction pattern having peaks at about 8.8, 13.5, 15.5, 15.9, 18.4, 20.4, 22.3, 22.8 and 25.8±0.2 degrees 2-theta; and combinations thereof.

Preferably the crystalline form XIV of dexLansoprazole has a crystalline purity of at least 80%, more preferably at least 90%, more preferably at least 95%, most preferably at least 99% by weight.

In another embodiment, the invention encompasses a process for preparing the crystalline Form XIV of dexLansoprazole comprising: dissolving dexLansoprazole in EtOH and ammonium hydroxide; adding diisopropyl ether to the solution; removing the resulting precipitate; and evaporating the filtrate from the solution to obtain a solid.

Preferably, the amount of ethanol is about 1 to about 10 ml per gram of dexLansoprazole, more preferably, about 2 to about 8 ml per gram of dexLansoprazole, more preferably, about 4 to about 6 ml per gram of dexLansoprazole, most preferably, about 5.2 ml per gram of dexLansoprazole.

Preferably, the ratio of ammonium hydroxide to ethanol is of about 1:6 to about 1:16.

Preferably, the ratio of diisopropyl ether to ethanol is of about 5:1 to about 15:1.

Preferably, after the diisopropyl ether addition, a stirring step is performed for a period of about 6 hours to about 24 hours, preferably of about 12 hours to about 24 hours.

Preferably, stirring is done at a temperature of about 5° C. to about −20° C., more preferably, at a temperature of about 5° C. to about 0° C.

Typically an initial precipitate is formed in the solution after the addition of diisopropyl ether and cooling of the solution, which is removed prior to evaporating the solvent from the solution. Preferably, removing this initial precipitate is done by filtration. Preferably, the obtained filtrate is dried by evaporation to obtain a precipitate.

The evaporation may be done under reduced pressure.

In yet another embodiment, the invention encompasses a process for preparing the crystalline Form V of dexLansoprazole comprising: dissolving dexLansoprazole in H₂O:EtOAc; adding water to the solution and cooling to obtain a precipitate.

In another embodiment, the invention encompasses a process for preparing the crystalline Form XIV of dexLansoprazole comprising: dissolving dexLansoprazole in H₂O:EtOAc; adding water to the solution; cooling to obtain a precipitate; and drying.

Preferably, the ratio of H₂O:EtOAc is about 1:5 to about 5:1, more preferably, about 1:1. Preferably, the amount of water added is about 10 volumes.

In the two processes described immediately above, cooling is preferably to a temperature of about −10° C. to a temperature of about −20° C., more preferably, to a temperature of about −20° C.

Preferably, cooling is performed while stirring. Preferably, the stirring is for about 12 hours to about 48 hours, more preferably, for about 14 hours.

Preferably, cooling is done for about 12 hours to about 48 hours, more preferably, for about 14 hours.

The drying may be done in a vacuum oven at a temperature of about 40° C. to about 50° C.

The invention also encompasses a process for drying Form V of dexLansoprazole to obtain Form XIV of dexLansoprazole. Preferably, Form V is obtained according to the process described above.

The drying may be done in a vacuum oven at a temperature of about 40° C. to about 50° C.

The obtained precipitate is further isolated. Typically, the isolation is by filtration.

The invention encompasses a process for preparing the crystalline Form IV of dexLansoprazole comprising: slurrying dexLansoprazole form II in a solvent selected from the group consisting of a mixture of H₂O:CH₂Cl₂ and isopropyl acetate to obtain a precipitate.

The invention encompasses a process for preparing the crystalline Form XIV of dexLansoprazole comprising: slurrying form II of dexLansoprazole in a mixture of H₂O and CH₂Cl₂ to obtain a precipitate; and drying.

Preferably, the ratio of H₂O:CH₂Cl₂ is about 1:5 to about 5:1, more preferably, about 1:1. Preferably, the amount of water added is about 10 volumes.

Preferably, the starting material is dexLansoprazole form II.

Preferably, the amount of isopropyl acetate is about 5 volumes to about 10 volumes, more preferably, about 7 volumes.

Preferably, the slurry is maintained while stirring. Preferably, stirring is for about 12 hours to about 48 hours, more preferably for about 12 hours to about 24 hours. Preferably, stirring is at about room temperature.

The drying may be done in a vacuum oven at a temperature of about 40° C. to about 50° C.

The invention also encompasses a process for drying Form IV of dexLansoprazole to obtain Form XIV of dexLansoprazole. Preferably, Form IV is obtained according to the process described above.

The drying may be done in a vacuum oven at a temperature of about 40° C. to about 50° C.

The obtained precipitate is further isolated. Typically, the isolation is by filtration.

In yet another embodiment, the invention encompasses a process for preparing the crystalline Form XIV of dexLansoprazole comprising: dissolving dexLansoprazole in a solvent selected from the group consisting of: 2-pentanol and chloroform; adding hexane to the solution to obtain a precipitate; and drying.

The solution may be stirred prior to the hexane addition of hexane. The stirring may be done over night.

Preferably, following the hexane addition, a stirring step is performed. Preferably, the stirring is for about 24 hours to about 72 hours, more preferably for about 44 hours to about 50 hours, even more preferably, for about 48 hours.

The obtained precipitate is further isolated. Typically, the isolation is by filtration.

The drying may be done in a vacuum oven at a temperature of about 40° C. to about 50° C. Preferably, drying is performed for about 12 hours to about 48 hours, more preferably, for about 16 to about 24 hours.

In yet another embodiment, the invention encompasses a process for preparing the crystalline Form XIV of dexLansoprazole comprising: slurrying dexLansoprazole form II in isopropylacetate to obtain a precipitate; and drying.

Preferably, the slurry is maintained while stirring. Preferably, the stirring is for about 12 hours to about 48 hours, more preferably for about 12 hours to about 24 hours. Preferably, the stirring is at about room temperature.

Preferably, the drying is done a temperature of about 40° C. to about 50° C.

The obtained precipitate is further isolated. Typically, the isolation is by filtration.

The present invention comprises 1) a pharmaceutical composition comprising any one, or combination of dexLansoprazole crystalline forms X, XI, XII, XIII or XIV described above and at least one pharmaceutically acceptable excipient; and 2) the use of any one, or combination, of the above-described dexLansoprazole crystalline forms X, XI, XII, XIII or XIV, in the manufacture of a pharmaceutical composition, wherein the pharmaceutical composition can be useful for the treatment or prevention of erosive oesophagitis and non-erosive gastroesophageal reflux.

The pharmaceutical composition of the present invention can be in solid or a non-solid form. If the pharmaceutical composition is in a non-solid form, any one, or combination, of the dexLansoprazole crystalline forms X, XI, XII, XIII or XIV are retained as solid(s) in the non-solid pharmaceutical composition e.g., as a suspension, foam, ointment and etc.

The pharmaceutical composition can be prepared by a process comprising combining any one, or combination of the above-described dexLansoprazole crystalline forms X, XI, XII, XIII or XIV with at least one pharmaceutically acceptable excipient. The dexLansoprazole crystalline forms X, XI, XII, XIII or XIV can be obtained by any of the processes of the present invention as described above.

The pharmaceutical composition can be used to make appropriate dosage forms such as tablets, powders, capsules, suppositories, sachets, troches, and lozenges.

Any one, or combination, of the above-mentioned dexLansoprazole crystalline forms X, XI, XII, XIII or XIV, particularly in a pharmaceutical composition and dosage form, can be used to treat or prevent erosive oesophagitis and non-erosive gastroesophageal reflux in a mammal such as a human, comprising administering a treatment effective amount of the one, or combination, of the dexLansoprazole crystalline forms X, XI, XII, XIII or XIV in the mammal. The treatment effective amount or proper dosage to be used can be determined by one of ordinary skill in the art, which can depend on the method of administration, the bioavailability, the age, sex, symptoms, and health condition of the patient, and the severity of the disease to be treated, etc.

Having described the invention with reference to certain preferred embodiments, other embodiments will become apparent to one skilled in the art from consideration of the specification. The invention is further defined by reference to the following examples describing in detail the preparation of dexLansoprazole dexLansoprazole crystalline forms of the invention. It will be apparent to those skilled in the art that many modifications, both to materials and methods, may be practiced without departing from the scope of the invention.

### Instruments

### Powder X-Ray Diffraction

For ARL Powder diffractometer (All XRD Figures except FIG. 13):

Sample: Spin/Oscillate mode
Range: 2-40 degrees two-theta
Scan mode: Continuous scan
Step size: 0.05 deg
Scan rate: 3 deg./min

For Philips X′Pert PRO Powder diffractometer (FIG. 13)

Samples are applied directly on silicon

Sample holder preparation PW1817/32 “zero background” holder

### Instrument Philips X'Pert PRO

### Goniometer PW3050/60

### Generator PW3040; 45 kV, 40 mA

X-Ray tube PW3373/00; Cu anode LFF
X-ray radiation λ(CuKα1)=1.540598 Å

### Temperature 295±5 K

### TGA/DSC

TGA and DSC analysis as shown in FIGS. 3, 4, 8 and 15 was performed using the following instruments:

Type of instrument: TGA/DSC 1 of Mettler-Toledo (FIGS. 8, and 15) or TA Instruments TGA 2959 (FIG. 4).

Heating range: 25-250 C; heating rate: 10° C./min,
Nitrogen flow 40 ml/min
Mass weight about 5 mg.

### DSC

DSC analysis of Form X as illustrated in FIG. 3 was performed using a TA Instruments Q1000

Heating range: 25-160 C; heating rate: 10° C./min,
Nitrogen flow 40 ml/min
Mass weight about 1 mg.

### EXAMPLES

### Example 1

### Preparation of Form X

R-(+)-Lansoprazole (4 g, 87.5% ee) was dissolved in EtOH 95% (16 mL) and aqueous ammonium hydroxide 25% (1 mL) was added. The resulting solution was stirred at room temperature for 30 min, whereupon precipitation occurred. The solids were removed by filtration and the filtrate was evaporated under reduced pressure to give an off-white solid (94% ee (enantiomeric excess)). The solid was dried in a vacuum oven at 25° C. for 16 h to give form X.

### Example 2

### Preparation of Form XI

To a 1 ml vial containing a magnetic stirrer bar was added R-(+)-Lansoprazole (20.1 mg, 94.6% ee), followed by the addition of ethyleneglycol (0.1 mL) whilst stirring. The vial was closed and the resulting clear solution was stirred at room temperature over night. Water (0.2 mL) was added to the solution, which led to precipitation. The resulting slurry was filtered immediately to give form XI.

### Example 3

### Preparation of Form XI

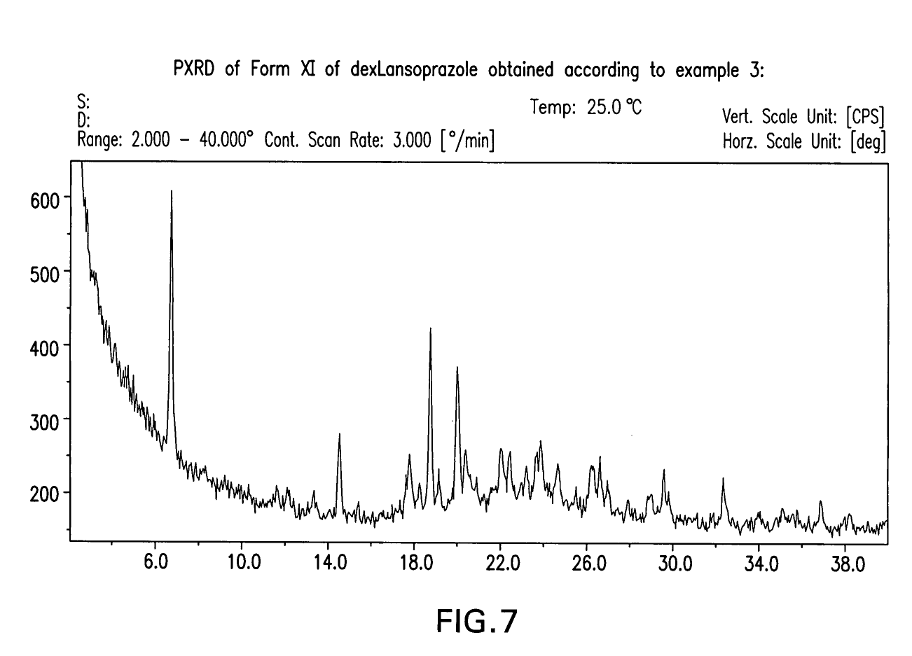

To a solution of R-Lansoprazole (0.020 g, 0.054 mmol) in ethylene glycol (0.2 mL) was added water (0.4 mL), which led to the formation of a precipitate. The mixture was heated to 80° C. and stirred at this temperature for 1 h, followed by cooling to room temperature. The resulting slurry was stirred overnight at room temperature. The solids were collected by filtration, without further drying, and identified by XRD as form XI, as presented in FIG. 7.

### Example 4

### Preparation of Form XI

To a solution of R-Lansoprazole (0.250 g, 0.677 mmol) in ethylene glycol (0.8 mL) was added hexane (0.6 mL). The resulting solution was cooled to −20° C. overnight. At this stage, no precipitation could be observed. The mixture was heated to room temperature after which water (0.8 mL) was added. The resulting slurry was stirred overnight at room temperature; the solids were filtered off and identified by XRD as form XI, as presented in FIG. 6.

### Example 5

### Preparation of Form XII

To a 1 ml vial containing a magnetic stirrer bar was added R-(+)-Lansoprazole (0.025 g, 94.6% ee), followed by the addition of propyleneglycol (0.1 mL) during stirring. The vial was closed and the resulting clear solution was stirred at room temperature over night. Water (0.2 mL) was added to the solution, which led to the formation of a milky-white suspension. Stirring was continued at room temperature for another day and then the resulting slurry was filtered to give form XII.

### Example 6

### Preparation of Form XIII

R-(+)-lansoprazole (2.0 g, 73% ee) was dissolved by stirring in absolute EtOH (8 mL, 4 Vol) at room temperature and ammonium hydroxide 25% (0.5 mL) was added to the solution. This led to the formation of a precipitate, which was filtered and the filtrate was evaporated to dryness to give (1.67 g, 76% ee). The above described procedure was repeated with 4 vol EtOH and ammonium hydroxide 25% (0.5 mL) and the resulting precipitate was removed from the mother liquor, which was concentrated in vacuum to give (1.65 g, 80% ee). To this material was added EtOH (6 mL) but no dissolution was observed. Subsequently, ammonium hydroxide 25% (1 mL) was added and the mixture was heated to 35-40° C. to allow dissolution. The resulting solution was cooled to room temperature and diisopropyl ether (50 mL) was added dropwise. Cooling to 0-(+5)° C. led to precipitation. The solids were separated from their mother liquors by filtration. HPLC analysis of both solid and filtrate suggested an optical purity level of 17% ee in the solid and 93.6% ee in the filtrate. The filtrate was evaporated under reduced pressure and dried in a vacuum oven at 25° C. for 16 h to give a solid (1.3 g, 94% ee) of Form XIII.

### Example 7

### Preparation of Form XIII of DexLansoprazole

R-(+)-lansoprazole (4.1 g, 87.5% ee), previously prepared using a known procedure and ensuing column chromatography, was dissolved in absolute EtOH (16 mL) at room temperature and 1 mL ammonium hydroxide (25%) was added. The resulting solution was cooled to 0-5° C. to afford crystallization. The solids were separated and the filtrate concentrated under reduced pressure to give “A” as a white-brown foam (3.13 g, 88% ee). The procedure was repeated with this material “A” using 6 mL EtOH and 1 mL ammonium hydroxide to give solid and filtrate. The latter was evaporated to dryness to give “B” (2.7 g, 90% ee). This material was subjected to a third precipitation cycle, this time using 12 mL EtOH and 1 mL ammonium hydroxide. The resulting solids were removed and the filtrate concentrated in vacuo to give “C” (2.5 g, 92% ee). At this stage the material was dissolved at room temperature in minimum amount EtOH (5 mL, 2 Vol) and 1 mL ammonium hydroxide was added, followed by dropwise addition of diisopropylether (50 mL). The resulting clear solution was cooled to between 0 and +5° C. while stirring, which led to the formation of crystals. The solids “D” (0.54 g, 99% ee) were separated from the mother liquor, which after evaporation led to a solid “E” of dexLansoprazole (1.93 g, 90.4% ee).

### Example 8

### Preparation of Form XIII

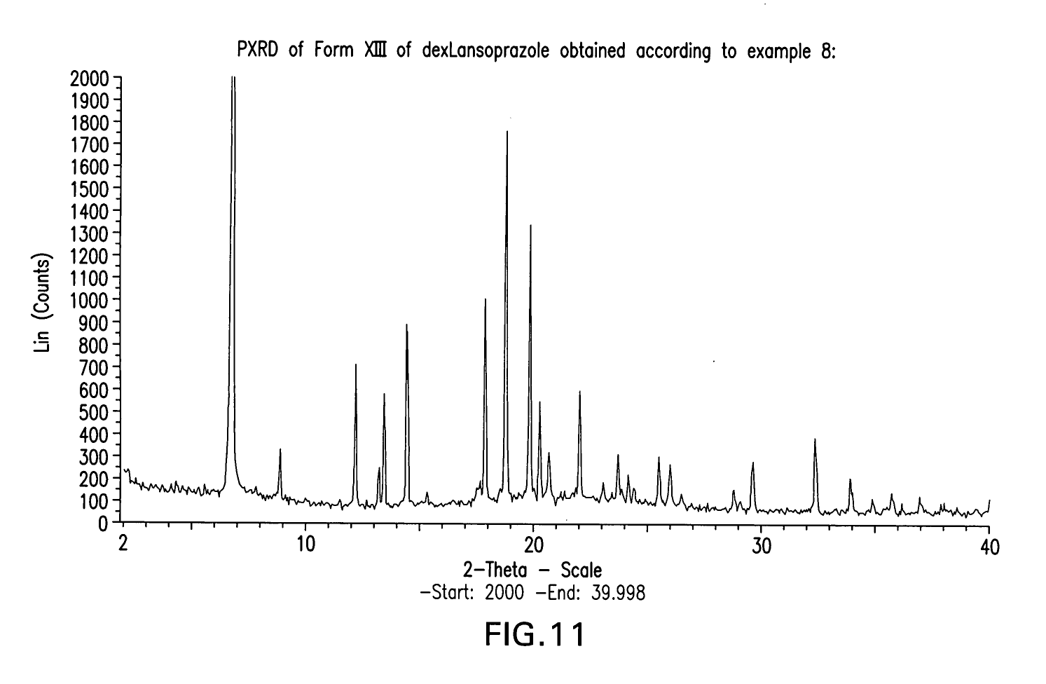

To a 1 ml vial was added R-Lansoprazole (0.050 g, 0.135 mmol). The open vial was inserted into a 10 ml vial, which was filled to ⅓ with ethanol. The larger vial was then closed with a screw cap. After 3 weeks standing at room temperature, the contents of the smaller vial were identified by powder XRD as form XIII, as presented in FIG. 11.

### Example 9

### Preparation of Form XIII

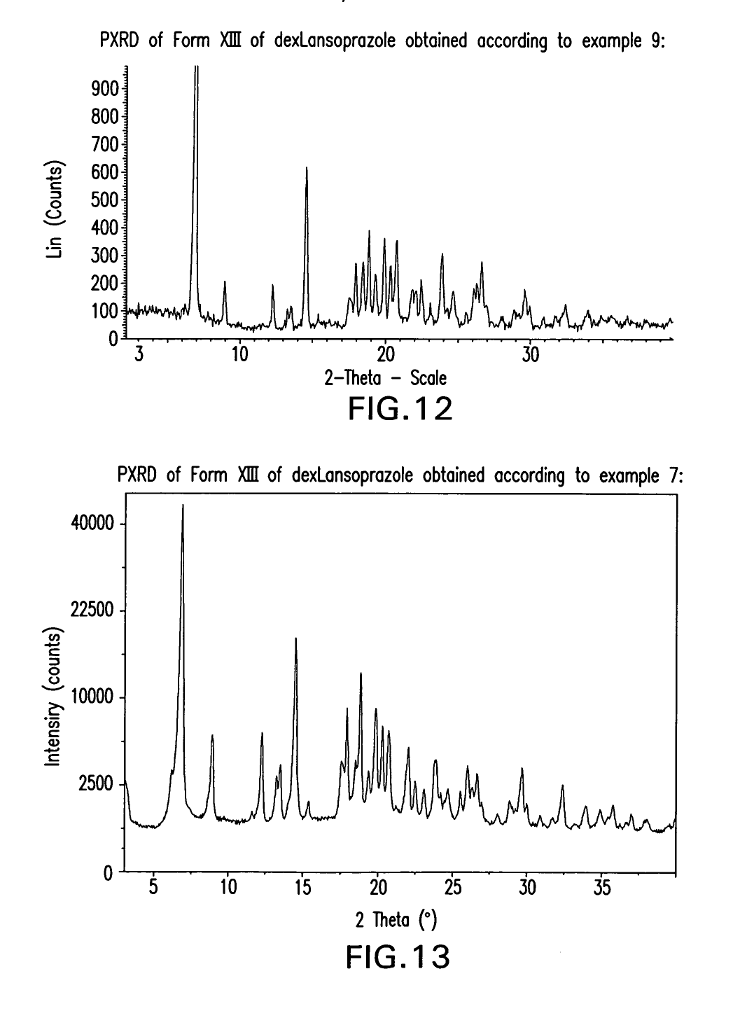

A drop of ethanol was added to about 50 mg of R-Lansoprazole that was placed in a mortar. The wet powder was strongly ground with a pestle for 1 minute. The product of the grinding was identified by XRPD as Form XIII of R-Lansoprazole, as presented in FIG. 12.

### Example 10

### Preparation of Form XIII

To 6.3 g of DLNP (mixture of form X and amorphous) was added portion-wise 3.5 mL EtOH absolute. (previously saturated with ammonia gas). The mixture was stirred with a spatula for 10 min at room temperature. The resultant yellow oily slurry was kept in a freezer at −18° C. for 1 week, leading to the formation of crystals in the oily residue. The crystals were isolated by centrifugation to give a solid XRD analysis suggested the material was a mixture of forms XIII+II, where form XIII is at least about 80% of the mixture.

### Example 11

### Preparation of Form XIII

To 3.6 g of DLNP (mixture of form XIV and amorphous) was added portion-wise 3.2 mL EtOH absolute. (previously saturated with ammonia gas) at 0-5° C. The resulting purple/pink mixture, which contained undissolved particles, was transferred to the freezer and kept at −18° C. for 1 week. The resulting crystals formed in the oily substance were isolated by filtration through a centrifuge to give a solid. XRD analysis identified the polymorphic forms as a mixture of XIII and III.

### Example 12

### Preparation of Form XIII

3.2 g DLNP (amorphous) was dissolved in 5 mL EtOH absolute at room temperature. Light turbidity was observed. Gradually the mixture became more viscous and turned into a thick slurry. Another 2 ml EtOH absolute was added (total EtOH V=7 mL). The mixture was seeded by portion-wise addition of 3.8 g DLNP. The resulting light slurry was stirred during 10-15 min and became highly viscous. The solids were isolated by filtration, but not washed. XRD of wet material wet was done immediately and suggested that the sample was a mixture of polymorphs II and XIII.

### Example 13

### Preparation of Form XIII

About 50 mg of dexLansoprazole form III was slurried in a saturated solution of dexLansoprazole in absolute ethanol at 23-25° C. Stirring was continued for 5 minutes and the resulting solids were evaporated at 23-25° C. and atmospheric pressure and subsequently analysed by XRD and identified as form XIII.

### Example 14

### Preparation of Form XIII

About 50 mg of dexLansoprazole form VI was exposed to ethanol vapour for 5 days at 23-25° C. and subsequently analysed by XRD and identified as form XIII.

### Example 15

### Preparation of Form XIII

About 50 mg of dexLansoprazole form X was exposed to ethanol vapour for 5 days at 23° C.-25° C. and subsequently analysed by XRD and identified as form XIII.

### Example 16

### Preparation of Form XIV

R-(+)-lansoprazole (3.65 g, 86.9% ee) was dissolved by stirring in absolute EtOH (19 mL, 5 Vol) at room temperature. Subsequently ammonium hydroxide 25% (2 mL) was added to the solution. From this stock solution was taken 2.5 mL, and to this sample was added diisopropyl ether (5 mL, 2 Vol). Stirring at 5° C. over night led to the formation of a precipitate, which was removed by filtration. The remaining filtrate was evaporated to dryness under reduced pressure to give (93.7% ee) an off-white solid as form XIV.

### Example 17

### Preparation of Form XIV

To a 1 ml vial was added 19 mg DLNP, followed by the addition of 0.2 ml of a mixture of H₂O:EtOAc (1:1 v/v), which afforded a clear solution at room temperature. Subsequently another 0.2 ml of H₂O was added, and the mixture was cooled to −20° C. for 14 h with stirring to obtain a precipitate. After filtration, the collected solid was dried in a vacuum oven at 40° C. to give Form XIV.

### Example 18

### Preparation of Form XIV

To a 1 ml vial was added 28 mg DLNP form II, followed by 0.2 ml of a mixture of H₂O:CH₂Cl₂ (1:1 v/v). The resulting slurry was stirred at room temperature over night. After filtration (which resulted in wet form IV of DLNP) and drying in a vacuum oven at 40° C. Form XIV was obtained.

### Example 19

### Preparation of Form XIV

To a 1 ml vial was added 21 mg DLNP, followed by 2-pentanol (0.2 ml) which resulted in a clear solution. The solution was stirred at room temperature over night. Hexane (0.2 ml) was added and stirring was continued for 2 days to allow the formation of a precipitate. After filtration and drying of the solids (wet form III of DLNP) in a vacuum oven at 40° C. Form XIV was obtained.

### Example 20

### Preparation of Form XIV

To a 1 ml vial was added 17 mg DLNP, followed by chloroform (0.2 ml). Stirring of the resulting clear solution was continued over night, after which hexane (0.2 ml) was added with a further 2 days of stirring. This resulted in the formation of a precipitate which was isolated through filtration. The solid (form III of DLNP) was dried under vacuum at 40° C. to give Form XIV.

### Example 21

### Preparation of Form XIV

To a 1 ml vial was added 23 mg DLNP form II, followed by isopropylacetate (0.2 ml). The resulting slurry was stirred over night at ambient temperature. After filtration, the collected solid (form III of DLNP) was dried in vacuo at 40° C. to afford Form XIV.

1. A crystalline form of DexLansoprazole characterized by data selected from the group consisting of: an X-ray powder diffraction pattern having peaks at about: 6.8, 12.2, 16.4, 17.8, 20.4 and 22.4±0.2 degrees 2-theta; and an X-ray powder diffraction pattern substantially as depicted in FIGS. 1 and 2; and combinations thereof.

2-4. (canceled)

5. The crystalline form of claim 1 further characterized by data selected from the group consisting of: an X-ray powder diffraction pattern having peaks at about: 6.8, 12.2, 14.4, 16.4, 17.8 and 20.4±0.2 degrees 2-theta; a DSC thermogram having an endothermic peak in the range of about 40° C. to about 90° C.; a weight loss when heating to a temperature of about 100° C. of less than about 2% as measured by TGA; a TGA pattern as depicted in FIG. 3; a DSC pattern as depicted in FIG. 4; and combinations thereof.

6. (canceled)

7. A crystalline form of DexLansoprazole characterized by data selected from the group consisting of: an X-ray powder diffraction pattern having peaks at about 8.9 and 10.2±0.2 degrees 2-theta and at least two more peaks selected from the group consisting of peaks at about: 13.2, 13.5, 15.5, 15.9, 18.4 and 22.3±0.2 degrees 2-theta; an X-ray powder diffraction pattern having peaks at about 15.5, 15.9, 20.4, 22.3 and 22.8; an X-ray diffraction pattern substantially as depicted in FIG. 14; and combinations thereof.

8-9. (canceled)

10. The crystalline form of claim 7, further characterized by data selected from the group consisting of: a DSC thermogram having endothermic peaks in a range of about 40° C. to about 60° C. and in the range of about 110° C. to about 140° C.; a DSC pattern as depicted in FIG. 15; an X-ray powder diffraction pattern having peaks at about 8.8, 10.2, 13.5, 15.9 and 18.4±0.2 degrees 2-theta; an X-ray powder diffraction pattern having peaks at about 8.8, 10.2, 13.2, 15.5 and 22.3±0.2 degrees 2-theta; an X-ray powder diffraction pattern having peaks at about 8.8, 13.5, 15.5, 15.9, 18.4, 20.4, 22.3, 22.8 and 25.8±0.2±0.2 degrees 2-theta; and combinations thereof.

11-17. (canceled)

18. A crystalline form of DexLansoprazole characterized by data selected from the group consisting of: an X-ray powder diffraction pattern having peaks at about 14.4 and 19.2±0.2 degrees 2-theta and at least two more peaks selected from the group consisting of peaks at about: 18.4, 18.8, 20.6, 23.8 and 26.6±0.2 degrees 2-theta; an X-ray powder diffraction pattern having peaks at about: 6.7, 12.2, 13.4, 14.4 and 17.9±0.2 degrees 2-theta; an X-ray powder diffraction pattern having peaks at about: 6.7, 12.2, 14.4, 18.8 and 20.7±0.2 degrees 2-theta; an X-ray diffraction pattern substantially as depicted in any one of FIGS. 10-13; and combinations thereof.

19-24. (canceled)

25. The crystalline form of claim 18, further characterized by data selected from a group consisting of: an X-ray powder diffraction pattern having peaks at about 14.4, 18.4, 19.2, 20.6 and 23.8±0.2 degrees 2-theta; an X-ray powder diffraction pattern having peaks at about 14.4, 18.4, 18.8, 19.2 and 26.6±0.2 degrees 2-theta; an X-ray powder diffraction pattern having peaks at about 6.7, 12.2, 13.4, 14.4, 17.9, 18.4, 18.8, 19.2, 19.8, 20.3 and 20.7±0.2 degrees 2-theta; and combinations thereof.

26-33. (canceled)

34. A crystalline form of DexLansoprazole characterized by data selected from the group consisting of: an X-ray powder diffraction pattern having peaks at about: 11.6 and 32.3±0.2 degrees 2-theta and at least three more peaks selected from the group consisting of peaks at about: 6.7, 12.1, 14.5, 18.7, 20.0, 22.0 and 23.6±0.2 degrees 2-theta; an X-ray powder diffraction pattern having peaks at about: 6.7, 14.5, 18.7 and 20.0±0.2 degrees 2-theta; an X-ray diffraction pattern substantially as depicted in any one of FIGS. 5-7; and combinations thereof.

35-38. (canceled)

39. The crystalline form of claim 34, further characterized by data selected from the group consisting of: a DSC thermogram having an endothermic peak in a range of 80° C.-150° C.; TGA pattern as depicted in FIG. 8; a DSC pattern as depicted in FIG. 8; an X-ray powder diffraction pattern having peaks at about 11.6, 18.7, 22.0, 23.6 and 32.3±0.2 degrees 2-theta; an X-ray powder diffraction pattern having peaks at about 11.6, 12.1, 14.5, 20.0 and 32.3±0.2 degrees 2-theta; an X-ray powder diffraction pattern having peaks at about 6.7, 14.5, 17.8, 18.7, 20.0, 22.0, 29.6, 32.3 and 36.8±0.2 degrees 2-theta; and combinations thereof.

40-42. (canceled)

43. A crystalline form of DexLansoprazole characterized by data selected from the group consisting of: an X-ray powder diffraction pattern having peaks at about 5.5, 13.2 and 19.7±0.2 degrees 2-theta and at least two more peaks selected from the group consisting of peaks at about: 7.0, 16.6, 17.9, 20.3, 21.2, 22.5 and 26.1±0.2 degrees 2-theta; an X-ray powder diffraction pattern having peaks at about 5.5, 13.2, 19.7 and 21.2±0.2 degrees 2-theta; an X-ray diffraction pattern substantially as depicted in FIG. 9; and combinations thereof.

44-45. (canceled)

46. The crystalline form of claim 43, further characterized by data selected from the group consisting of: an X-ray powder diffraction pattern having peaks at about 5.5, 7.0, 13.2, 16.6 and 19.7±0.2 degrees 2-theta; an X-ray powder diffraction pattern having peaks at about 5.5, 13.2, 19.7, 21.2 and 22.5±0.2 degrees 2-theta; an X-ray powder diffraction pattern having peaks at about 5.5, 7.0, 13.2, 16.6, 19.7, 20.3, 21.2, 22.5 and 26.1±0.2 degrees 2-theta; and combinations thereof.

47. (canceled)

48. A pharmaceutical formulation comprising a therapeutically effective amount of at least one of the forms of dexLansoprazole as defined in any of claims 1, 5, 7, 10, 18, 25, 34, 39, 43, or 46, and at least one pharmaceutically acceptable excipient.

49. A process for preparing a pharmaceutical formulation comprising combining at least one of the forms of dexLansoprazole as defined in any of claims 1, 5, 7, 10, 18, 25, 34, 39, 43, or 46, with at least one pharmaceutically acceptable excipient.

50. Method of treating or preventing erosive oesophagitis and non-erosive gastroesophageal reflux comprising administering a pharmaceutical formulation comprising a therapeutically effective amount of at least one of the forms of dexLansoprazole as defined in any of claims 1, 5, 7, 10, 18, 25, 34, 39, 43, or 46, and at least one pharmaceutically acceptable excipient to a patient in need thereof.
